(19) United States
(12) Patent Application Publication
(10) Pub. No.: US 2017/0020453 A1
(43) Pub. Date: Jan. 26, 2017

(54) BIOLOGICAL INFORMATION DETECTION DEVICE

(71) Applicant: SEIKO EPSON CORPORATION, Tokyo (JP)

(72) Inventors: Ryoichi NAKAGAWA, Matsumoto-shi (JP); Tetsu NAKAYAMA, Matsumoto-shi (JP); Yoshihiro HASHIZUME, Ina-shi (JP)

(73) Assignee: SEIKO EPSON CORPORATION, Tokyo (JP)

(21) Appl. No.: 15/217,905

(22) Filed: Jul. 22, 2016

(30) Foreign Application Priority Data
Jul. 23, 2015 (JP) 2015-145572

(51) Int. Cl. A61B 5/00; A61B 5/024

(52) U.S. Cl. CPC A61B 5/681; A61B 5/6843; A61B 5/6844; A61B 5/02427; A61B 5/7445

(57) ABSTRACT

A biological information detection device includes a case that is provided with a sensor that detects biological information about a subject, a band that secures the case on the subject, and an adjustment mechanism that pulls the band in a first direction when a rotary member that is supported by the case has been rotated, the first direction being a direction in which the side of the case that is situated opposite to the subject comes in contact with the subject.

Japanese Patent Application No. 2015-145572 filed on Jul. 23, 2015, is hereby incorporated by reference in its entirety.

### BACKGROUND

A biological information detection device that is worn by the user using a band (belt) or the like is widely known. Examples of such a biological information detection device include a biological information detection device (pulse wave measurement device) that is worn on the wrist of the user using a band, and detects pulse wave information based on the pulsation of the blood vessel at a position around the wrist.

Since the measurement accuracy of such a biological information detection device is affected by the wearing state, it is important to implement an appropriate wearing state (i.e., appropriately secure the biological information detection device on the user at a given position).

A structure that utilizes a hole and a locking member is widely used for a wristwatch and the like as a structure that adjusts the degree of fastening of the band. Specifically, a plurality of holes are formed in the band, and the locking member (rod-like member) provided to a buckle is inserted into one of the plurality of holes to adjust (determine) the degree of fastening (inner diameter) of the band.

JP-A-2010-110634 discloses a biological information detection device (biological information measurement device) that includes an elastic member that is connected to a band by means of sewing. JP-A-2010-110634 discloses that the elastic member expands and contracts in the longitudinal direction of the band so that the biological information detection device comes in contact with the wrist of the user.

### SUMMARY

According to one aspect of the invention, there is provided a biological information detection device comprising:

a case that is provided with a sensor that detects biological information about a subject;

a band that secures the case on the subject; and

an adjustment mechanism that moves the band in a first direction when a rotary member that is supported by the case has been rotated, the first direction being a direction in which a side of the case that is situated opposite to the subject comes in contact with the subject.

### BRIEF DESCRIPTION OF THE DRAWINGS

FIG. 1 illustrates a configuration example of a common biological information detection device.

FIG. 2 is a view illustrating an adjustment operation according to one embodiment of the invention.

FIG. 3 is a schematic view illustrating an adjustment mechanism.

FIG. 4 is another schematic view illustrating an adjustment mechanism.

FIG. 5 is a schematic view illustrating a release mechanism.

FIG. 6 is a plan view and a side view illustrating a biological information detection device according to one embodiment of the invention.

FIG. 7 is a perspective view illustrating a biological information detection device according to one embodiment of the invention.

FIG. 8 is a plan view and a side view illustrating a rotary bezel.

FIG. 9 is a perspective view illustrating a rotary bezel.

FIG. 10 is a plan view and a side view illustrating a ratchet mechanism.

FIG. 11 is a perspective view illustrating a ratchet mechanism.

FIG. 12 is a plan view illustrating a ratchet mechanism in a state in which the ratchet mechanism is secured on a case.

FIG. 13 is a plan view and a side view illustrating a release mechanism that includes a release button.

FIG. 14 is a perspective view illustrating a release mechanism that includes a release button.

FIG. 15 is a schematic external view illustrating a biological information detection device when an adjustment mechanism is provided separately from a case.

FIG. 16 is a schematic external view illustrating a biological information detection device when a fitting hole and a locking member are not provided.

### DESCRIPTION OF EXEMPLARY EMBODIMENTS

According to one embodiment of the invention, there is provided a biological information detection device comprising:

a case that is provided with a sensor that detects biological information about a subject;

a band that secures the case on the subject; and

an adjustment mechanism that moves the band in a first direction when a rotary member that is supported by the case has been rotated, the first direction being a direction in which a side of the case that is situated opposite to the subject comes in contact with the subject.

According to one aspect of the invention, the band is moved in the first direction by rotating the rotary member so that the sensor comes in contact with the subject. According to this configuration, it is possible to appropriately adjust the wearing state (i.e., the degree of fastening of the band and the degree of contact of the sensor with the subject) by adjusting the moving amount of the band by means of the rotation of the rotary member, and detect the biological information with high accuracy, for example.

In the biological information detection device,

the adjustment mechanism may include a restriction mechanism that restricts movement of the band in a second direction that is a direction opposite to the first direction.

This makes it possible to suppress an unintentional movement of the band, for example.

In the biological information detection device,

the adjustment mechanism may include a wire that is connected to the band, and is at least either moved or deformed due to the rotation of the rotary member.

This makes it possible to move the band by means of the movement or the deformation of the wire.

In the biological information detection device,

the rotary member may be a rotary bezel, and

the restriction mechanism may be a ratchet mechanism that engages with the rotary bezel, and restricts the movement of the band in the second direction.

This makes it possible to use the rotary bezel as the rotary member, and use the ratchet mechanism as the restriction mechanism.

In the biological information detection device,

the adjustment mechanism may include a wire that connects the ratchet mechanism and the band, and may be configured so that the ratchet mechanism is rotated in a first rotation direction in conjunction with the rotary bezel when the rotary bezel is rotated in the first rotation direction so that the wire moves the band in the first direction.

According to this configuration, it is possible to move the wire and the band connected to the wire by rotating the ratchet mechanism (for which the rotation direction is restricted) in conjunction with the rotary bezel.

In the biological information detection device,

the ratchet mechanism may disengage from the rotary bezel so that the band can be moved in the second direction when the rotary bezel has been moved upward in a direction that intersects a rotation plane of the rotary bezel.

According to this configuration, it is possible to perform an adjustment that moves the band in the second direction so as to reduce the degree of fastening by moving the rotary bezel upward so that the rotary bezel disengage from the ratchet mechanism.

In the biological information detection device,

the rotary bezel may be movable in a direction that intersects a rotation plane of the rotary bezel so that the band can be moved in the second direction.

According to this configuration, it is possible to move the rotary bezel in order to retract the band so as to reduce the degree of fastening.

In the biological information detection device,

the band may include a plurality of fitting holes, and a locking member that is fitted into a fitting hole among the plurality of fitting holes to lock the band, and

a moving amount of the band corresponding to one tooth of a gear that is included in the ratchet mechanism may be smaller than an interval between the plurality of fitting holes.

According to this configuration, it is possible to implement a finer adjustment as compared with an adjustment using a fitting hole and a locking member by utilizing the ratchet mechanism.

In the biological information detection device,

the adjustment mechanism may be provided to the case.

This makes it possible to provide the adjustment mechanism to the case.

In the biological information detection device, the band may include a first band and a second band, the case may be provided between a first end of the first band and a first end of the second band, and the adjustment mechanism may be provided between a second end of the first band that differs from the first end, and a second end of the second band that differs from the first end.

This makes it possible to provide the adjustment mechanism at a position differing from the case.

In the biological information detection device, the band may include a plurality of fitting holes, and a locking member that is fitted into a fitting hole among the plurality of fitting holes to lock the band, and the adjustment mechanism may implement the fastening of the band between a state in which the locking member is fitted into a first fitting hole among the plurality of fitting holes, and a state in which the locking member is fitted into a second fitting hole among the plurality of fitting holes that is situated adjacent to the first fitting hole.

According to this configuration, it is possible to implement a finer adjustment as compared with an adjustment using the fitting holes and the locking member by utilizing the adjustment mechanism.

In the biological information detection device, the adjustment mechanism may be able to move the band in the first direction at an interval smaller than an interval between the plurality of fitting holes.

According to this configuration, it is possible to implement a finer adjustment as compared with an adjustment using the fitting holes and the locking member by utilizing the adjustment mechanism.

Exemplary embodiments of the invention are described below. Note that the following exemplary embodiments do not in any way limit the scope of the invention laid out in the claims. Note also that all of the elements described below in connection with the following exemplary embodiments should not necessarily be taken as essential elements of the invention.

### 1. Method

A method used in connection with the exemplary embodiments of the invention is described below. A biological information detection device that is worn on a given part of the user (subject) using a band is known (see above). An example in which the biological information detection device is a band-type (wristwatch-type) biological information detection device that is worn on the wrist of the user is described below. Note that the biological information detection device may be a device that is worn on another part (e.g., neck or ankle) of the user.

When using such a wearable biological information detection device, the user must wear the biological information detection device in an appropriate state. For example, a pulse wave measurement device that utilizes a photoelectric sensor is known as the biological information detection device. The pulse wave appears as a change in blood volume, and the photoelectric sensor (pulse wave sensor) measures the pulse wave by detecting a change in blood volume in the measurement target part. More specifically, the photoelectric sensor includes a light-emitting section that applies light to the subject, and a light-receiving section that receives the reflected light from tissue (blood vessel in a narrow sense). The light-emitting section may be implemented by an LED, and the light-receiving section may be implemented by a photodiode (PD), for example. Note that the acquisition of pulse wave information using the photoelectric sensor is widely known in the art, and detailed description thereof is omitted.

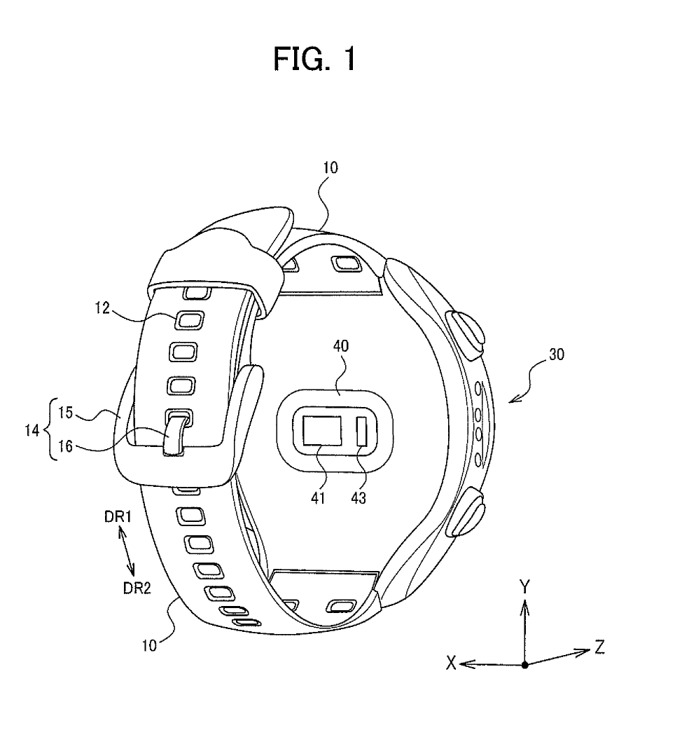

When the pulse wave information is acquired using the photoelectric sensor, light that has been applied by the light-emitting section and reflected by the blood vessel of the subject (i.e., reflected light) is detected by the light-receiving section, and light other than the reflected light is considered to be a noise component. Therefore, it is desirable that the biological information detection device be configured to suppress the incidence of disturbance light on the light-receiving section. For example, the biological information detection device illustrated in FIG. 1 has a configuration in which a biological information detection section 40 (sensor unit) is provided to a main body (case 30) so as to be situated on the side of the subject, and the sensor unit is situated between tissue and the main body in a state in which the user wears the biological information detection device. According to this configuration, since the main body can be used as a shielding member, it is possible to suppress a situation in which disturbance light (e.g., sunlight or illumination light) is incident on the light-receiving section (i.e., it is possible to suppress at least a situation in which disturbance light is directly incident on the light-receiving section).

When using such a configuration, however, the incidence of disturbance light can be suppressed only when the sensor unit (main body) comes in contact with the subject. For example, when the sensor unit has come off the subject (i.e., when a space has been formed between the sensor unit and the subject) even momentarily, disturbance light is incident on the light-receiving section through the space formed between the sensor unit and the subject. In this case, it is difficult to accurately measure the pulse wave information. Therefore, it is necessary to tightly secure the biological information detection device on the subject to such an extent that the sensor unit does not come off the subject.

Since the pulse wave information is information that reflects the active state and the health state of the user, it is very important to measure the pulse wave information during exercise in addition to measuring the pulse wave information during rest or sleep.

However, the user makes a large motion during exercise (e.g., the user swings the arms during running). This means that it is likely that the sensor unit comes off the subject during exercise as compared with during rest or the like. Therefore, it is necessary to more tightly fasten the band during exercise as compared with during rest or the like.

If the band is fastened too tightly, however, the user may feel pain in the area with which the band comes in contact, or skin irritation may occur through sweating. Although it may be possible to relieve such pain or irritation by changing the material that forms the band, the shape of the band, and the like, it is effective to prevent a situation in which the band is fastened too tightly. Specifically, it is strongly desired to prevent a situation in which the band is fastened too tightly. Note that load is applied to the subject from the band when the band is fastened. The load applied to the subject from the band is determined by the relationship between the inner diameter of the biological information detection device (including the band) and the outer diameter of the part (wrist) of the subject on which the biological information detection device is worn.

As described above, it is necessary to design the biological information detection device so that the user can appropriately wear the biological information detection device. More specifically, it is important to design the biological information detection device so that the user can fasten the band to such an extent that the biological information can be measured with high accuracy, but pain or the like does not occur.

The degree of fastening of the band may be adjusted using a fitting hole and a locking member that are widely used for a wristwatch, for example. FIG. 1 illustrates a specific example. FIG. 1 is a perspective view illustrating a biological information detection device in a state in which a band 10 is secured using a fitting hole 12 and a locking member 16 (viewed from the side of the band 10 so that the side of a case 30 that comes in contact with the subject when the biological information detection device is worn is observed). The biological information detection device illustrated in FIG. 1 has a configuration in which a plurality of fitting holes 12 are formed in the band 10, and the user inserts the locking member 16 provided to a buckle 14 into one of the plurality of fitting holes 12 when wearing the biological information detection device. As illustrated in FIG. 1, the plurality of fitting holes 12 are provided along the longitudinal direction of the band 10. When a fitting hole 12 among the plurality of fitting holes 12 that is situated closer to the end (see DR1) of the band 10 is selected, the inner diameter of the band 10 increases when the user wears the biological information detection device, and the degree of fastening of the band 10 decreases. When a fitting hole 12 among the plurality of fitting holes 12 that is situated closer to the main body (see DR2) is selected, the inner diameter of the band 10 decreases when the user wears the biological information detection device, and the degree of fastening of the band 10 increases.

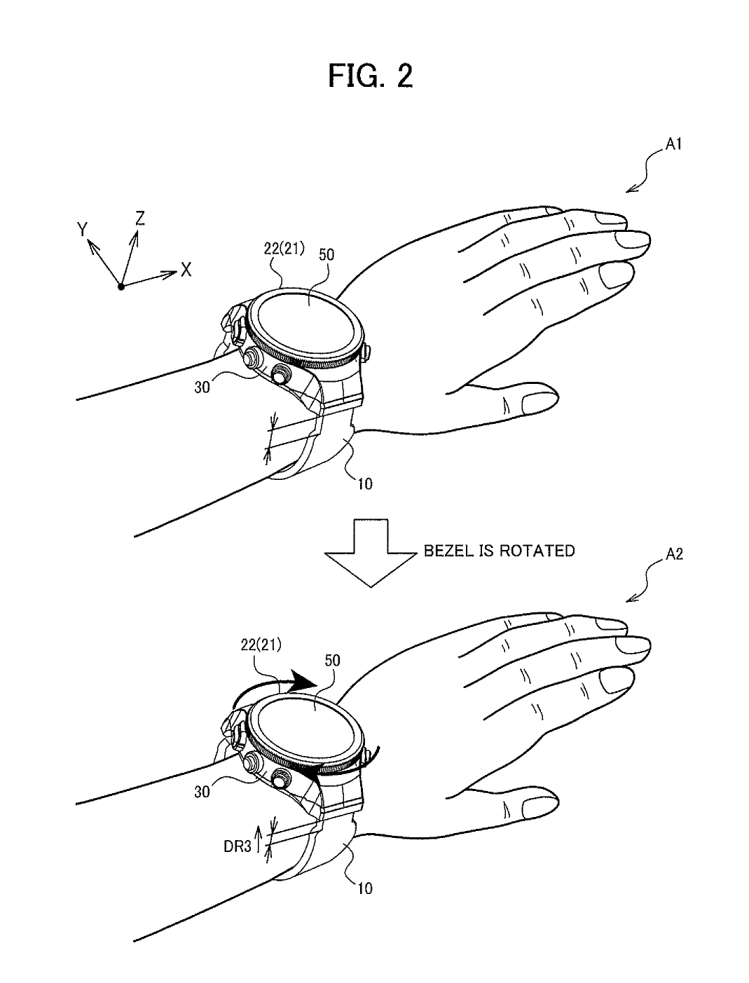

Note that a direction and the like may be defined using a given coordinate system for convenience of explanation. Specifically, a coordinate system is set with respect to the case 30 of the biological information detection device (see FIG. 1), and a direction that intersects a display section 50 (that corresponds to the face of a normal wristwatch) and extends from the back side toward the front side (display plane) of the display section 50 is referred to as a positive Z-axis direction. The positive Z-axis direction corresponds to the direction from the subject toward the case 30 in a state in which the subject wears the biological information detection device. Two axes that are orthogonal to the Z-axis are referred to as an X-axis and a Y-axis. The Y-axis corresponds to the direction in which the band 10 is connected (attached) to the case 30. In the example illustrated in FIG. 1, the band 10 is respectively connected to the end point of the case 30 in the positive Y-axis direction and the end point of the case 30 in the negative Y-axis direction. The above coordinate system is also applied to FIG. 2 and the like. Note that the direction in which the band 10 is connected (attached) to the case 30 may be set to be the Y-axis direction, the direction that is orthogonal to the Y-axis direction and extends along the normal to the surface of a biological information detection section 40 that comes in contact with the body of the user (subject) may be set to be the X-axis direction, the direction orthogonal to the Y-axis direction and the Z-axis direction may be set to be the X-axis direction.

When adjusting the degree of fastening using the fitting holes 12, however, it is impossible to adjust the degree of fastening more finely than the interval between the fitting holes 12. Therefore, a situation may occur in which the degree of fastening is too high when a given fitting hole 12 is selected, and is too low when the fitting hole 12 that is situated adjacent to the fitting hole 12 (in the direction DR1 in the example illustrated in FIG. 1) is selected. It is possible to adjust the degree of fastening more finely by reducing the interval between the fitting holes 12. However, this is not practical.

Specifically, when the interval between the fitting holes 12 is reduced, breakage of the band 10 may occur due to a decrease in strength, for example. Since the fitting holes 12 are normally formed to have a size of about 1 mm to about 2 mm, the interval between the fitting holes 12 must be set to about 4 mm to about 4.5 mm in order to ensure that the band 10 exhibits sufficient strength, for example. Therefore, the degree of fastening of the band 10 is necessarily adjusted in units of about 4 mm to about 4.5 mm.

The degree of fastening of the band 10 may be adjusted to be appropriate for the target user by cutting the band 10 corresponding to the thickness of the wrist of the user. In this case, however, it is difficult to adjust the degree of fastening after the band 10 has been cut. The thickness of the wrist of the user may change in the long term along with a change in weight or physique, and may change in the short term due to swelling, for example. It is impossible to deal with such a change if the band 10 is cut.

According to the method disclosed in JP-A-2010-110634, an elastic member is provided so that the biological information detection device can be fitted to the user. According to the method disclosed in JP-A-2010-110634, however, since the elastic member is connected by means of sewing, a problem occurs in terms of strength. According to the method disclosed in JP-A-2010-110634, the degree of fastening (inner diameter) of the band is determined by the material that forms the elastic member and the thickness of the wrist of the user, and it is impossible to flexibly adjust the degree of fastening of the band.

As described above, it is normally unnecessary to use the same degree of fastening in both a resting state (or a sleep state) and an exercise state. Specifically, it is necessary to increase the degree of fastening of the band 10 in an exercise state since the sensor unit easily comes off the subject. On the other hand, since it is considered that the motion of the arm is not large in a resting state, it is possible to detect the biological information with sufficient accuracy even when the degree of fastening of the band 10 is lower than that in an exercise state. Moreover, it is possible to suppress or reduce the occurrence of pain, irritation, or the like by reducing the degree of fastening of the band 10 (see above). Specifically, since the state of the band 10 that makes it possible to accurately detect the biological information changes corresponding to the situation, it is desirable that the state (degree of fastening) of the band 10 can be flexibly adjusted corresponding to each user.

In view of the above problems, several embodiments of the invention propose a biological information detection device that makes it possible to easily implement a fine adjustment of the band 10. More specifically, a biological information detection device according to one embodiment of the invention includes a case 30 that is provided with a biological information detection section 40 that detects biological information about a subject, a band 10 that secures the case 30 on the subject, and an adjustment mechanism 20 that moves the band 10 in a first direction when a rotary member 21 has been rotated, the first direction being a direction in which the side of the case 30 that is situated opposite to the subject comes in contact with the subject.

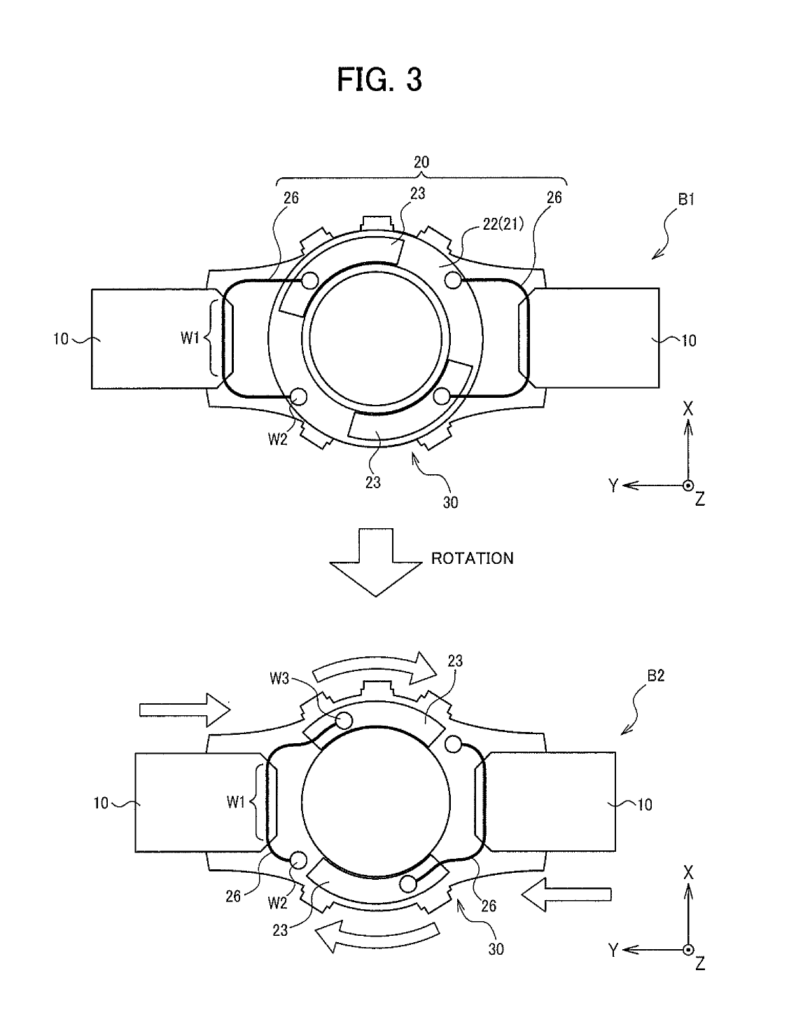

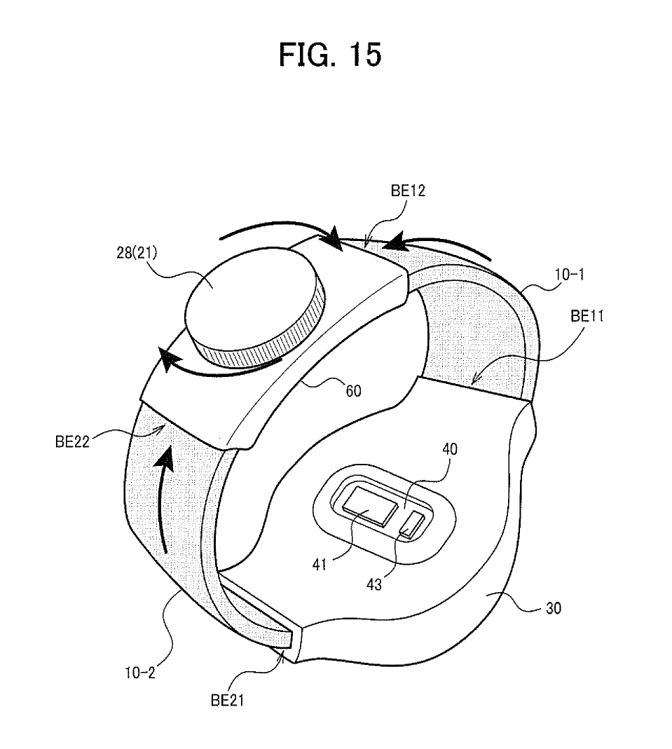

The rotary member 21 is a member that is supported by the case 30 (e.g., rotary bezel 22 described later with reference to FIG. 2 and the like). Note that a rotary operation section 28 illustrated in FIG. 15 may also be used as the rotary member 21. The adjustment mechanism 20 (pulling mechanism, winding mechanism, or take-up mechanism) corresponds to a ratchet mechanism 23 and a wire 26 (as described later with reference to FIG. 3 and the like). The adjustment mechanism 20 may include the rotary member 21 (rotary bezel 22) (see FIG. 3). The movement (pulling, winding, or taking up) of the band 10 in the first direction that is implemented by the adjustment mechanism 20 (pulling mechanism, winding mechanism, or take-up mechanism) means that the band 10 is moved using the adjustment mechanism 20 in the direction in which the degree of fastening increases (i.e., the band 10 is moved toward the adjustment mechanism 20 in a narrow sense). In other words, the length of part of the band 10 that is exposed from the case 30 is reduced, or the overlapping area of the case 30 and the band 10 when viewed along the Z-axis direction is increased.

The movement (retraction, release of winding, or release of taking up) of the band 10 in a second direction (described later) means that the band 10 is moved in the direction opposite to the first direction (i.e., in the direction away from the adjustment mechanism 20 in a narrow sense). In other words, the length of part of the band 10 that is exposed from the case 30 is increased, or the overlapping area of the case 30 and the band 10 when viewed along the Z-axis direction is reduced. Note that the movement of the band 10 in the first direction may be hereinafter referred to as “pulling in the first direction” or “pulling”, and the movement of the band 10 in the second direction may be hereinafter referred to as “retraction in the second direction” or “retraction”.

FIG. 2 is a schematic view illustrating the operation of the biological information detection device according to one embodiment of the invention. FIG. 2 is a view illustrating the biological information detection device that is worn by the user viewed from the side where the display section 50 is provided. FIG. 2 illustrates the biological information detection device in a state in which the rotary member 21 is rotated (see A1), and also illustrates the biological information detection device in a state in which the rotary member 21 has been rotated (see A2). FIG. 2 illustrates an example in which the rotary member 21 is used as the bezel (rotary bezel 22) of the case 30. Note that the modification described later with reference to FIG. 15 may also be employed.

As illustrated in FIG. 2, the biological information detection device according to one embodiment of the invention is configured so that the adjustment mechanism 20 pulls the band 10 in the first direction when the rotary member 21 is rotated in a given direction (clockwise in FIG. 2). The first direction refers to the direction in which the inner diameter of the band 10 decreases, and corresponds to the direction DR3 illustrated in FIG. 2. For example, when the adjustment mechanism 20 is provided inside the case 30, the first direction is the direction from the band 10 toward the case 30, and the adjustment mechanism 20 introduces part of the band 10 into the case 30.

According to this configuration, since the degree of fastening of the band 10 can be adjusted corresponding to the degree of rotation of the rotary member 21, it is possible to flexibly adjust the band 10. For example, the degree of fastening of the band 10 may be roughly adjusted using the fitting holes 12 and the locking member 16, and a fine adjustment may be made by rotating the rotary member 21. It is possible to adjust the degree of fastening of the band 10 corresponding to the situation by relatively decreasing the rotation amount of the rotary member 21 in a resting state, and relatively increasing the rotation amount of the rotary member 21 in an exercise state, for example.

A schematic configuration example of the biological information detection device according to one embodiment of the invention will be described first, and an example of a specific structure of each section of the biological information detection device will then be described.

### 2. Configuration Example of Biological Information Detection Device

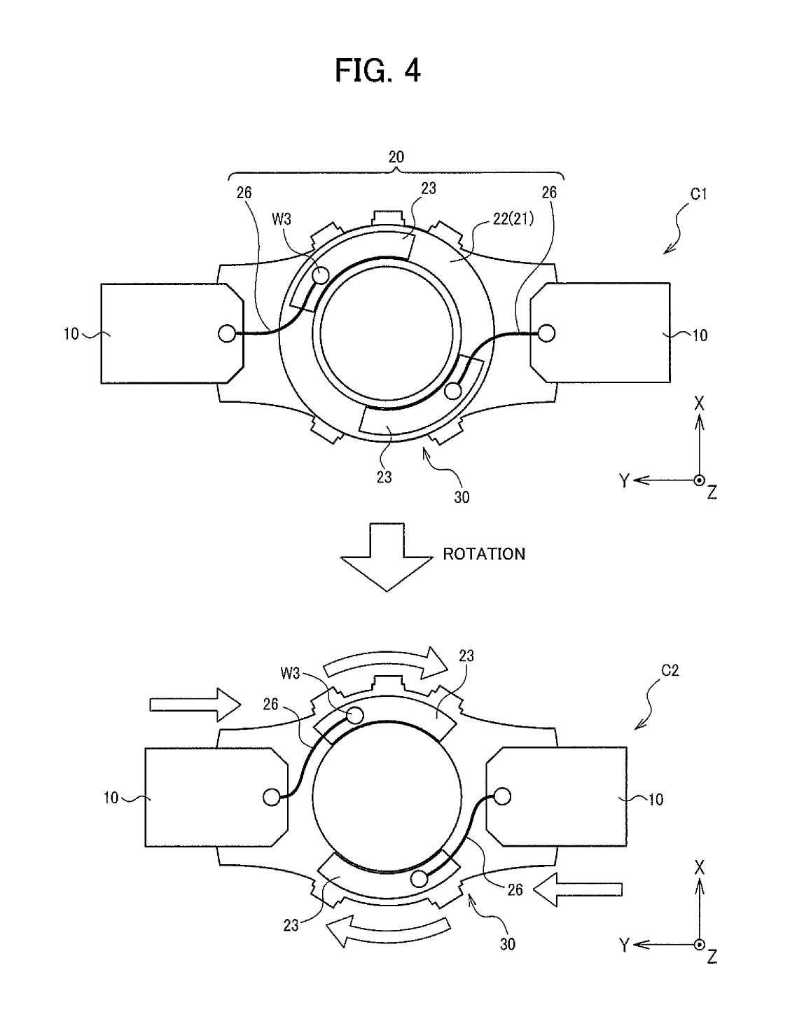

A configuration example of the biological information detection device is described below. A general configuration example of the biological information detection device is described below with reference to FIGS. 1 and 2. The band adjustment using the rotary member 21 is described later. Note that FIG. 1 is a view illustrating a known biological information detection device that does not utilize the adjustment mechanism 20 according to one embodiment of the invention. Since the biological information detection device according to one embodiment of the invention can be configured in the same manner as the biological information detection device illustrated in FIG. 1 except for the adjustment mechanism 20, FIG. 1 is also used to describe to the configuration of the biological information detection device according to one embodiment of the invention (except for the adjustment mechanism 20). An operation example of the adjustment mechanism 20 when the rotary member 21 is rotated will then be described with reference to FIGS. 3 and 4.

### 2.1 Configuration Example of Band-Type Biological Information Detection Device

As illustrated in FIGS. 1 and 2, the biological information detection device includes the band 10, the case 30, and the biological information detection section 40 (sensor or sensor unit). The case 30 is secured on the band 10. The sensor unit is provided to the case 30. The biological information detection device also includes a processing section 200 (not illustrated in the drawings). The processing section 200 is provided to the case 30, and detects biological information based on a detection signal output from the sensor unit.

The band 10 is wound around the wrist of the user when the user wears the biological information detection device. The band 10 is provided with the fitting holes 12 (band holes) and the buckle 14. The buckle 14 includes a band insertion section 15 and the locking member 16 (protrusion). When the user wears the biological information detection device on the wrist, the user inserts one end of the band 10 into the band insertion section 15 of the buckle 14, and inserts the locking member 16 of the buckle 14 into one of the fitting holes 12 of the band 10. The user can adjust the inner diameter of the band, and adjust the pressing force applied by the sensor unit (i.e., the pressing force applied to the surface of the wrist) by inserting the locking member 16 into an appropriate fitting hole 12. Note that the band 10 may be provided with a connection section 60 (buckle) illustrated in FIG. 15 instead of the buckle 14.

The case 30 corresponds to the main body of the biological information detection device. Various constituent parts (e.g., sensor unit and processing section 200) of the biological information detection device are provided inside the case 30. Specifically, the case 30 is a housing that holds these constituent parts. The case 30 may include a top case 34 and a bottom case 36, for example. Note that the case 30 need not necessarily be separated into the top case 34 and the bottom case 36. For example, the entire case 30 may be integrally formed.

The display section 50 may be provided to the case 30. The display section 50 displays various display screens. For example, the display section 50 may be implemented by a liquid crystal display, an organic EL display, or the like. Although FIG. 2 and the like illustrate an example in which the display section 50 is used as the user interface, the configuration is not limited thereto. For example, a light-emitting section may be used instead of (or together with) the display section 50. In this case, the light-emitting section is implemented by an LED or the like, and refers to an information display light-emitting section that differs from a light-emitting section 43 of the sensor unit.

A terminal (not illustrated in the drawings) may be provided to the case 30. When the biological information detection device is fitted to a cradle (not illustrated in the drawings), the terminal of the cradle and the terminal of the case 30 are electrically connected. A secondary battery (battery) provided to the case 30 can thus be charged. Note that a microUSB terminal may be provided to the biological information detection device, and the secondary battery may be charged using a microUSB cable, for example.

The biological information detection section 40 (sensor unit) detects biological information (e.g., pulse wave) about the subject. The sensor unit includes a light-receiving section 41 and a light-emitting section 43, for example. Note that the biological information that is detected by the biological information detection device according to one embodiment of the invention is not limited to a pulse wave (pulse rate). The biological information detection device may be a device that detects biological information (e.g., oxygen saturation in blood, body temperature, or heart rate (heartbeat)) other than a pulse wave. In such a case, a sensor other than a photoelectric sensor may be provided as the sensor unit.

### 2.2 Outline of Adjustment Mechanism

FIG. 3 is a schematic view illustrating the adjustment mechanism 20 according to one embodiment of the invention. FIG. 3 is a view illustrating the biological information detection device viewed from the side situated in the positive Z-axis direction. FIG. 3 illustrates the state of the adjustment mechanism 20 before the rotary member 21 is rotated (see B1), and the state of the adjustment mechanism 20 after the rotary member 21 has been rotated (see B2). As illustrated in FIG. 3, the adjustment mechanism 20 of the biological information detection device according to one embodiment of the invention includes the wire 26 that is connected to the band 10, and is at least either moved or deformed due to the rotation of the rotary member 21 (rotary bezel 22 in the example illustrated in FIG. 3). The term “movement” used herein in connection with the wire 26 refers to a relative change in position with respect to the case 30, for example. The term “deformation” used herein in connection with the wire 26 refers to a change in shape. For example, the term “deformation” used herein in connection with the wire 26 refers to a change in the bending position or the bending angle of the wire 26, or a change in the length (expansion/shrinkage) of the wire 26.

In the example illustrated in FIG. 3, the wire 26 is provided so that part W1 of the wire 26 passes through part (one end in a narrow sense) of the band 10, one end W2 of the wire 26 is secured on the case 30 at a given position, and the other end W3 of the wire 26 is pulled in the direction along the first direction in conjunction with the rotation of the rotary member 21. In the example illustrated in FIG. 3, when the other end W3 of the wire 26 is moved in the first direction (i.e., the direction from the band 10 to the case 30) due to the rotation of the rotary member 21, part of the wire 26 that is situated closer to the one end W2 passes through the band 10 (i.e., corresponds to the part W1). As a result, the distance between the one end W2 and the part W1 of the wire 26 decreases, and the band 10 that is connected to the part W1 of the wire 26 is also pulled in the first direction. In the example illustrated in FIG. 3, the band 10 that is connected to the wire 26 is pulled by allowing at least part (W3) of the wire 26 to be moved due to the rotation of the rotary member 21, and the wire 26 to be deformed due to the movement of the at least part (W3) of the wire 26. Although an example in which two bands 10 that are provided on either side (end) of the case 30 are pulled in the first direction (i.e., the direction toward the case 30) is described below, only one of the two bands 10 may be pulled in the first direction, for example.

According to the above configuration, it is possible to pull the band 10 that is connected to the wire 26 by pulling the wire 26 using the rotary member 21. The wire 26 can be implemented using a relatively thin member. Therefore, it is possible to implement the adjustment mechanism 20 using a smaller space as compared with the case where the rotation of the rotary member 21 is used to pull the band 10 by utilizing a mechanical structure (e.g., gear).

Note that the wire 26 need not necessarily be connected as illustrated in FIG. 3. As illustrated in FIG. 4, one end of the wire 26 may be connected to part (end in a narrow sense) of the band 10, and the other end of the wire 26 may be pulled in the direction along the first direction in conjunction with the rotation of the rotary member 21, for example. According to the configuration illustrated in FIG. 4, it is also possible to pull the band 10 that is connected to the wire 26 by pulling the wire 26 using the rotary member 21. Note that FIG. 4 is a view illustrating the biological information detection device viewed from the side situated in the positive Z-axis direction. FIG. 4 illustrates the state of the adjustment mechanism 20 before the rotary member 21 is rotated (see C1), and the state of the adjustment mechanism 20 after the rotary member 21 has been rotated (see C2).

As described above, the biological information detection device according to one embodiment of the invention is configured so that the band 10 is pulled in the first direction (i.e., the direction in which the inner diameter of the band 10 decreases) by rotating the rotary member 21. It is desirable that the biological information detection device also include a mechanism that maintains the band 10 in a pulled state in addition to the mechanism that pulls the band 10 in the first direction in view of practical use. Specifically, when appropriate biological information can be detected in a state in which the band 10 has been pulled, it may be impossible to detect appropriate biological information if the pulled state is canceled. For example, if the band 10 is easily retracted (i.e., an appropriate state is easily canceled) when a force that retracts the band 10 in the second direction (i.e., the direction in which the inner diameter of the band 10 increases) has been applied, it is difficult to detect the biological information in a stable manner.

Therefore, it is desirable that the adjustment mechanism 20 according to one embodiment of the invention include a restriction mechanism that restricts the movement (retraction) of the band 10 in the second direction that is opposite to the first direction. Note that the movement (retraction) of the band 10 in the second direction means that the band 10 is moved in the direction away from the adjustment mechanism 20 in a narrow sense. In other words, the movement (retraction) of the band 10 in the second direction means that the restriction by the restriction mechanism is canceled. It may be considered that the term “retraction” used herein means that a pulled state (wound state) is released.

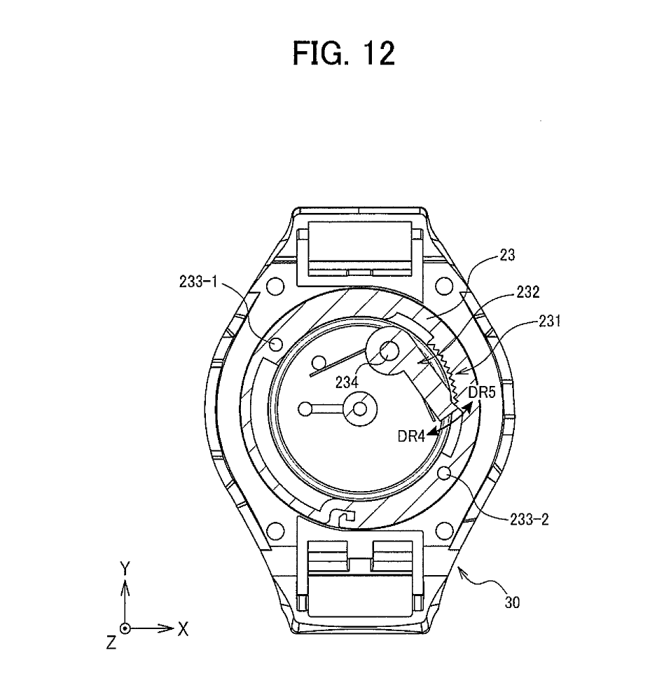

More specifically, the restriction mechanism may be a ratchet mechanism 23 that restricts the movement of the band 10 in the second direction. Note that the ratchet mechanism 23 is a mechanism that is widely known in the art. A ratchet mechanism having an arbitrary structure may be used as the ratchet mechanism 23. For example, the ratchet mechanism 23 is a mechanism that includes a gear 231 and a pawl 232 (described later with reference to FIG. 12).

In the examples illustrated in FIGS. 3 and 4, the rotation direction of the ratchet mechanism 23 is limited. Specifically, the ratchet mechanism 23 can be rotated clockwise, but cannot be rotated counterclockwise (i.e., the counterclockwise rotation of the ratchet mechanism 23 is prohibited). According to this configuration, it is possible to pull the band 10 in the first direction by connecting the wire 26 (the other end W3 of the wire 26 in a narrow sense) to the ratchet mechanism 23, and rotating the ratchet mechanism 23 clockwise. Since the counterclockwise rotation of the ratchet mechanism 23 is prohibited, the wire 26 is prohibited from returning to the non-pulled state. As a result, the retraction of the band 10 in the second direction is prohibited, and it is possible to maintain the band 10 in an appropriate state (pulled state).

It is also possible to easily adjust the pulling amount (moving amount) of the band 10 in the first direction (in addition to prohibiting the retraction of the band 10 in the second direction) by utilizing the ratchet mechanism 23. When the ratchet mechanism 23 that includes the gear 231 and the pawl 232 (described later with reference to FIG. 12) is used, it is necessary to rotate the gear 231 by applying a certain amount of force when the pawl 232 engages with the gear 231, and the pawl 232 is forced to be depressed due to the rotation of the gear 231. When the pawl 232 has been sufficiently depressed due to the rotation of the gear 231 so as to disengage from the gear 231, the gear 231 can be rotated without being restricted by the pawl 232. When the pawl 232 has engaged with the next tooth, it is necessary to apply a certain amount of force in order to rotate the gear 231.

According to this configuration, the rotation amount of the ratchet mechanism 23 can be adjusted corresponding to one tooth of the gear even when rotation in the forward direction occurs. As illustrated in FIGS. 3 and 4, the pulling width of the band 10 is determined by the rotation amount of the ratchet mechanism 23. Specifically, the pulling amount of the band 10 can be easily adjusted finely (i.e., corresponding to one tooth of the gear 231 in a narrow sense) by utilizing the ratchet mechanism 23. Therefore, it is possible to finely adjust the degree of fastening of the band 10.

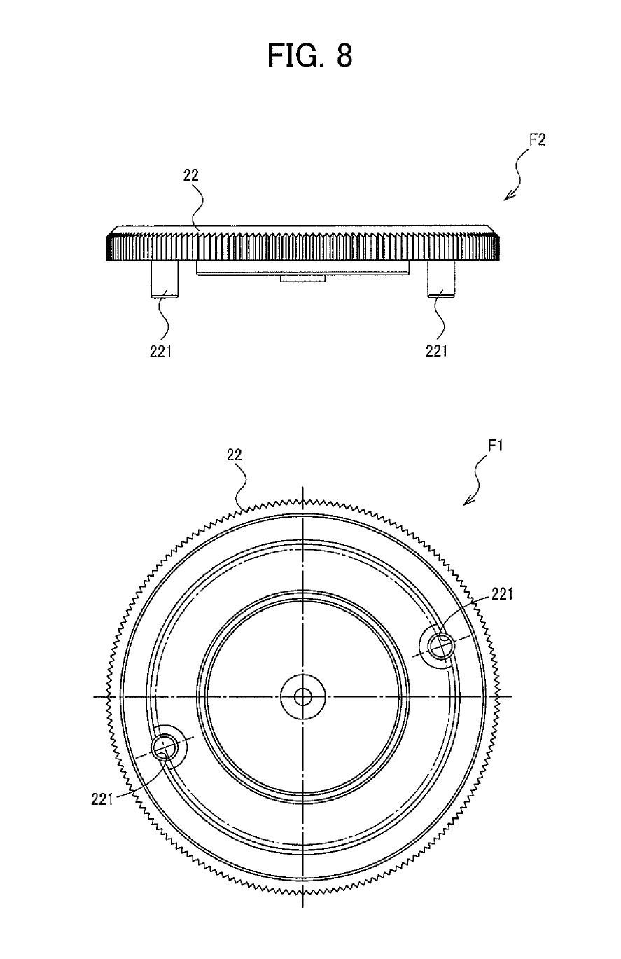

As illustrated in FIGS. 3 and 4, the rotary member 21 according to one embodiment of the invention may be the rotary bezel 22. In this case, the restriction mechanism may be the ratchet mechanism 23 that engages with the rotary bezel 22, and restricts the movement (retraction) of the band 10 in the second direction. For example, the rotary bezel 22 may have protrusions (221-1, 221-2), and the ratchet mechanism 23 may have recesses (233-1, 233-2) that engage with the protrusions (described later with reference to FIGS. 8 to 11). Note that the term “bezel” used herein refers to a structure that is widely used for a wristwatch and the like. The bezel is a member that forms the frame (edge). The bezel provided to a wristwatch has a ring-like shape and forms the frame of the face. The bezel provided to the biological information detection device according to one embodiment of the invention is a member that forms the frame of the case 30 (display section 50 in a narrow sense). The term “rotary bezel” used herein refers to a bezel that is configured to be rotatable. Specifically, the term “rotary bezel” used herein refers to a bezel that is configured to be rotatable in (along) a rotation plane that corresponds to (coincides with in a narrow sense) the face or the plane of the display section. In the example illustrated in FIG. 2 and the like, the rotary bezel 22 is a member that forms the frame of the display section 50, and is rotated in (along) a rotation plane that corresponds to the plane of the display section 50. The ratchet mechanism is a mechanism that restricts the motion (operation) direction to one direction. For example, the ratchet mechanism is implemented by securing a gear and a pawl on a given member. In one embodiment of the invention, the ratchet mechanism 23 is implemented by securing a member that includes the gear 231 and the pawl 232 on the case 30 (as described later with reference to FIG. 12).

In this case, since the ratchet mechanism 23 engages with the rotary bezel 22, the ratchet mechanism 23 is rotated due to the rotation of the rotary bezel 22. Since the reverse rotation (i.e., the counterclockwise rotation in the example illustrated in FIG. 3) of the ratchet mechanism 23 is prohibited as described above, the reverse rotation of the rotary bezel 22 is also prohibited in a state in which the ratchet mechanism 23 engages with the rotary bezel 22.

As described above, the adjustment mechanism 20 may include the wire 26 that connects the ratchet mechanism 23 and the band 10, and may be configured so that the ratchet mechanism 23 is rotated in the first rotation direction in conjunction with the rotary bezel 22 when the rotary bezel 22 is rotated in the first rotation direction so that the wire 26 moves (pulls) the band 10 in the first direction. The expression “in conjunction with” used herein in connection with the rotary bezel 22 and the ratchet mechanism 23 means that the rotation (rotational movement) of the rotary bezel 22 and the rotation (rotational movement) of the ratchet mechanism 23 have a relationship. Specifically, the expression “in conjunction with” used herein in connection with the rotary bezel 22 and the ratchet mechanism 23 means that the ratchet mechanism 23 is rotated in the first rotation direction due to the rotation of the rotary bezel 22 in the first rotation direction. The expression “in conjunction with” used herein in a narrow sense in connection with the rotary bezel 22 and the ratchet mechanism 23 may mean that, when the rotary bezel 22 is rotated in the first rotation direction by a given rotation amount, the ratchet mechanism 23 is rotated in the first rotation direction by the given rotation amount. This relationship can be implemented by designing the rotary bezel 22 and the ratchet mechanism 23 so as to be enable to engage with each other (see above).

The term “first rotation direction” used herein refers to the moving direction (rotation direction) of the ratchet mechanism 23 in which the rotation of the ratchet mechanism 23 is not prohibited. In the example illustrated in FIG. 3, the term “first rotation direction” refers to the clockwise rotation direction.

According to this configuration, it is possible to implement the adjustment mechanism 20 according to one embodiment of the invention by utilizing the rotary bezel 22, the ratchet mechanism 23, and the wire 26 in combination. Since the rotary bezel 22 is normally provided at a position corresponding to the frame of the case 30 (main body) (e.g., a position that surrounds the display section 50 (see FIG. 2)), the user can easily operate the rotary bezel 22 in a state in which the user wears the biological information detection device. When the rotary bezel 22 is provided at a position corresponding to the frame of the case 30, the size of the rotary bezel 22 can be relatively increased. For example, it is possible to easily implement the rotary bezel 22 having a size close to the maximum diameter of the case 30. Therefore, the user can easily operate the rotary bezel 22, and easily and finely adjust the degree of fastening of the band 10 by finely adjusting the rotation amount of the rotary bezel 22.

When the restriction mechanism is provided, it is also necessary to cancel (release) the restriction by the restriction mechanism. Otherwise, it is impossible to loosen the band 10 (retract the band 10 in the second direction). It is often necessary to loosen the band 10 (e.g., when the user gets rest after exercise).

For example, the restriction by the restriction mechanism can be released (canceled) by implementing a positional relationship in which the gear 231 and the pawl 232 that form the ratchet mechanism 23 do not engage with each other. More specifically, the restriction by the restriction mechanism may be released by moving the gear 231 to a position at which the gear 231 does not engage with the pawl 232. When the restriction by the restriction mechanism has been released, the band 10 can be pulled in the first direction, and retracted in the second direction. When the restriction by the restriction mechanism has been released, the band 10 is basically retracted sufficiently in the second direction to loosen the band 10, and then pulled in the first direction to implement readjustment. It is desirable to pull the band 10 in the first direction in a state in which the restriction by the restriction mechanism has been resumed. According to this configuration, it is possible to accurately and finely adjust the pulling width of the band 10 (see above).

More specifically, the rotary bezel 22 may be movable in the direction that intersects the rotation plane of the rotary bezel 22 so that the band 10 can be moved (retracted) in the second direction. This means that the positional relationship between the rotary bezel 22 and the case 30 can be relatively changed in the direction that intersects the rotation plane of the rotary bezel 22 at least when retracting the band 10. For example, the rotary bezel 22 may be fixed with respect to the case 30 as long as the rotary bezel 22 can protrude in the positive Z-axis direction when retracting the band 10. For example, the rotary bezel 22 may be designed so that the rotary bezel 22 is moved in the direction that intersects the rotation plane when a force equal to or larger than a given value has been applied in the direction that intersects the rotation plane, and is not moved when the magnitude of the force is less than the given value, or when a force has been applied in a direction that differs from the direction that intersects the rotation plane, or when a force other than gravity is not applied. In other words, the ratchet mechanism 23 may disengage from the rotary bezel 22 so that the band 10 can be moved (retracted) in the second direction when the rotary bezel 22 has been moved upward in the direction that intersects the rotation plane of the rotary bezel 22.

The direction that intersects the rotation plane refers to the direction that perpendicularly intersects the rotation plane in a narrow sense. Note that the direction that intersects the rotation plane is not limited thereto. For example, the direction that intersects the rotation plane may be a direction that almost perpendicularly intersects the rotation plane (e.g., a direction for which the difference in angle from the direction that perpendicularly intersects the rotation plane is equal to or smaller than a given angle threshold value). Since the rotary bezel 22 is normally provided at the position illustrated in FIG. 2, the rotation plane of the rotary bezel 22 corresponds to the XY plane that is the display plane of the display section 50, and the direction that intersects the rotation plane corresponds to the Z-axis direction. The direction in which the rotary bezel 22 can be moved is the positive Z-axis direction taking account of the interference of the rotary bezel 22 with other members that form the case 30.

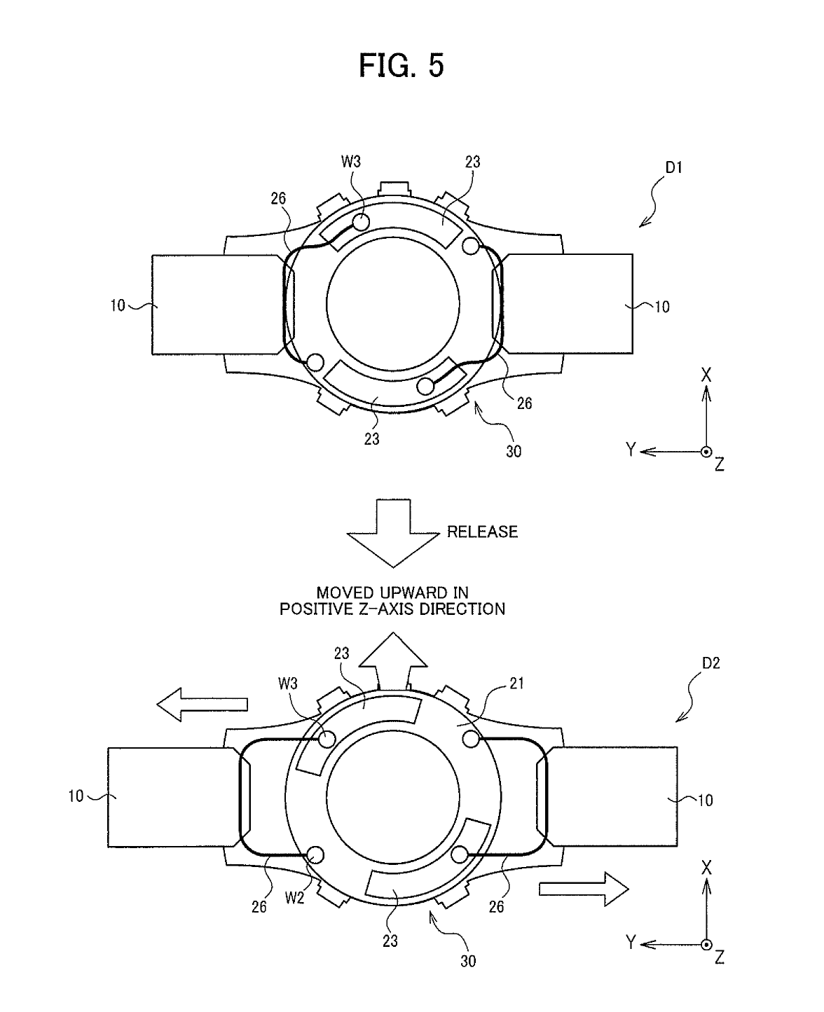

FIG. 5 is a schematic view illustrating the release mechanism. FIG. 5 is a view illustrating the biological information detection device viewed from the side situated in the positive Z-axis direction. FIG. 5 illustrates the release mechanism in a state in which the release operation is not performed (see D1), and the release mechanism in a state in which the release operation is performed (see D2). The ratchet mechanism 23 and the rotary bezel 22 engage with each other (i.e., the ratchet mechanism 23 (and the rotary bezel 22) can be rotated only in one rotation direction) in a normal state (i.e., a state in which the restriction by the restriction mechanism is enabled).

For example, the position of the pawl 232 of the ratchet mechanism 23 is determined by the rotary bezel 22 when the ratchet mechanism 23 and the rotary bezel 22 engage with each other. More specifically, the gear 231 and the pawl 232 of the ratchet mechanism 23 are set to have a positional relationship in which the gear 231 and the pawl 232 can engage with each other when the ratchet mechanism 23 and the rotary bezel 22 engage with each other, and are set to have a positional relationship in which the gear 231 and the pawl 232 cannot engage with each other when the ratchet mechanism 23 and the rotary bezel 22 do not engage with each other.

A specific structure may be designed in various ways. For example, the position of the pawl 232 of the ratchet mechanism 23 in the Z-axis direction may be changed corresponding to the state of the rotary bezel 22. In this case, when the rotary bezel 22 is moved from a state in which the ratchet mechanism 23 and the rotary bezel 22 do not engage with each other to a state in which the ratchet mechanism 23 and the rotary bezel 22 engage with each other, the pawl 232 is moved downward in the negative Z-axis direction so that the pawl 232 and the gear 231 can engage with each other. A mechanism is provided that moves the pawl 232 in the positive Z-axis direction when the rotary bezel 22 is moved in the positive Z-axis direction so that the ratchet mechanism 23 and the rotary bezel 22 do not engage with each other. This mechanism can be implemented using various means (e.g., spring). When the position of the pawl 232 and the position of the gear 231 in the positive Z-axis direction do not coincide with each other, the pawl 232 and the gear 231 cannot engage with each other, and the restriction by the ratchet mechanism 23 is disabled. According to this configuration, since whether or not to effect the restriction by the restriction mechanism can be determined corresponding to whether or not the rotary bezel 22 and the ratchet mechanism 23 engage with each other, it is possible to implement the above release mechanism. Alternatively, a cam surface may be provided to part of the rotary bezel 22, and a cam follower surface that guides the pawl 232 in the XY plane in a given direction due to the movement of the cam surface may be provided to part of the pawl 232. In this case, when the rotary bezel 22 is moved so as to disengage from the ratchet mechanism 23, the pawl 232 is guided by the cam surface and the cam follower surface in the direction in which the pawl 232 engages with the gear 231 (e.g., the direction from the center of the case 30 toward the outside). A mechanism is provided that moves the pawl 232 in the direction in which the pawl 232 cannot engage with the gear 231 (e.g., the direction toward the center of the case 30) when the rotary bezel 22 has disengaged from the ratchet mechanism 23, and no force (force that guides the pawl 232 in the above direction) is applied to the pawl 232 through the cam surface.

Note that the biological information detection device according to one embodiment of the invention is configured to make it possible to implement a finer adjustment as compared with the adjustment using the fitting hole 12 and the locking member 16 (see FIG. 1). In other words, the biological information detection device according to one embodiment of the invention has a configuration in which the band 10 includes a plurality of fitting holes 12, and the locking member 16 that is fitted into a fitting hole among the plurality of fitting holes 12 to lock the band 10, and the adjustment mechanism 20 implements the fastening of the band 10 between a state in which the locking member 16 is fitted into a first fitting hole, and a state in which the locking member 16 is fitted into a second fitting hole that is situated adjacent to the first fitting hole.

According to this configuration, it is possible to roughly adjust the band 10 using the fitting hole 12 and the locking member 16, and more finely adjust the band 10 using the adjustment mechanism 20. Specifically, it is possible to appropriately adjust the band 10 using two mechanisms. The fitting holes 12 and the locking member 16 can be configured in the same manner as illustrated in FIG. 1 since it is unnecessary to take account of a fine adjustment. Since it suffices that the adjustment mechanism 20 be able to pull the band 10 within a range equal to or smaller than the interval between the fitting holes 12 (in a narrow sense), it is unnecessary to increase the pulling amount.

Specifically, it suffices that the adjustment mechanism 20 be able to move (pull) the band 10 in the first direction (i.e., adjust the state of the band 10 secured on the case 30) at an interval smaller than the interval between the fitting holes 12. More specifically, it suffices that the moving width (pulling width or minimum pulling amount) of the band 10 by the adjustment mechanism 20 be smaller than the interval between the fitting holes 12. For example, the moving amount (pulling amount) of the band 10 that corresponds to one tooth of the gear 231 included in the ratchet mechanism 23 may be set to be smaller than the interval between the fitting holes 12.

Since it is easy to implement rotation control corresponding to one tooth of the gear by utilizing the ratchet mechanism 23 (see above), the minimum pulling amount of the band 10 can be set to be the pulling amount corresponding to one tooth of the gear. When the minimum pulling amount is smaller than the interval between the fitting holes 12, a finer adjustment as compared with the adjustment using the fitting holes 12 and the locking member 16 can be implemented using the adjustment mechanism 20. For example, when the interval between the fitting holes 12 is about 4 mm to about 4.5 mm, the minimum pulling amount achieved by utilizing the ratchet mechanism 23 can be set to about 0.5 mm. This makes it is possible to implement a finer adjustment.

### 3. Specific Example of Biological Information Detection Device

A specific example of each section of the biological information detection device according to one embodiment of the invention is described below with reference to the drawings. Note that the structure of each section of the biological information detection device is not limited to those described below. Various modifications and variations may be made.

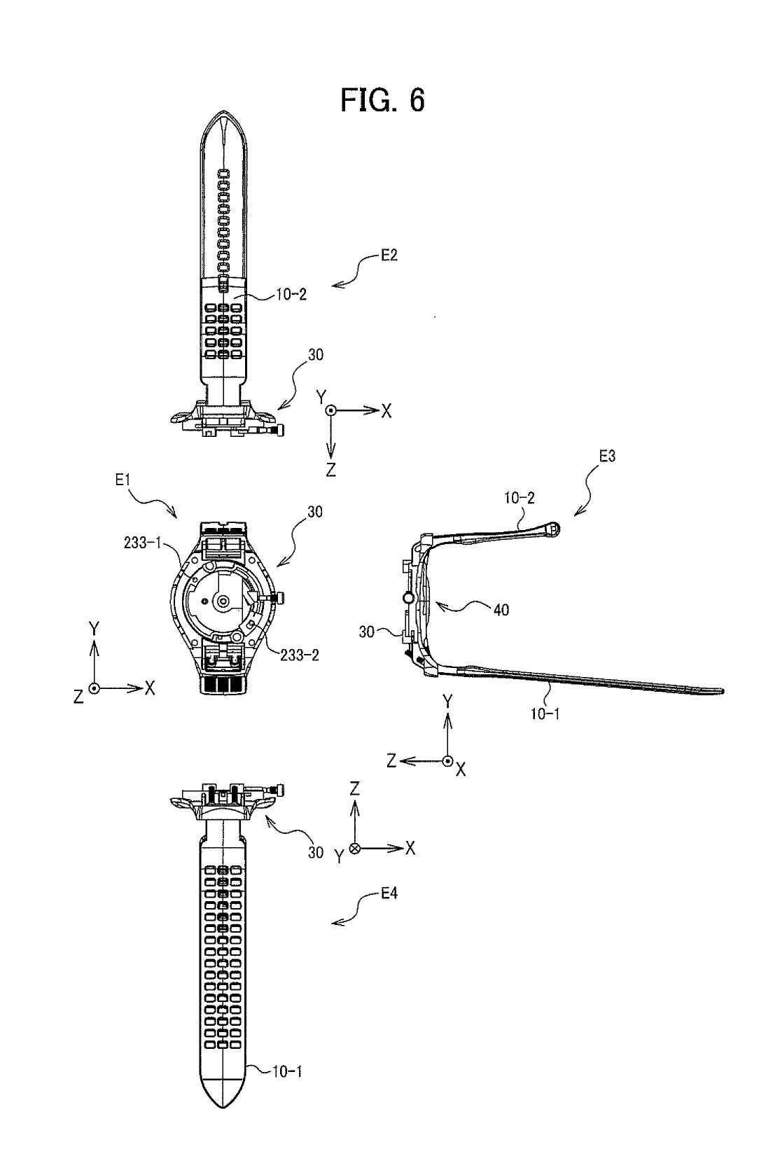

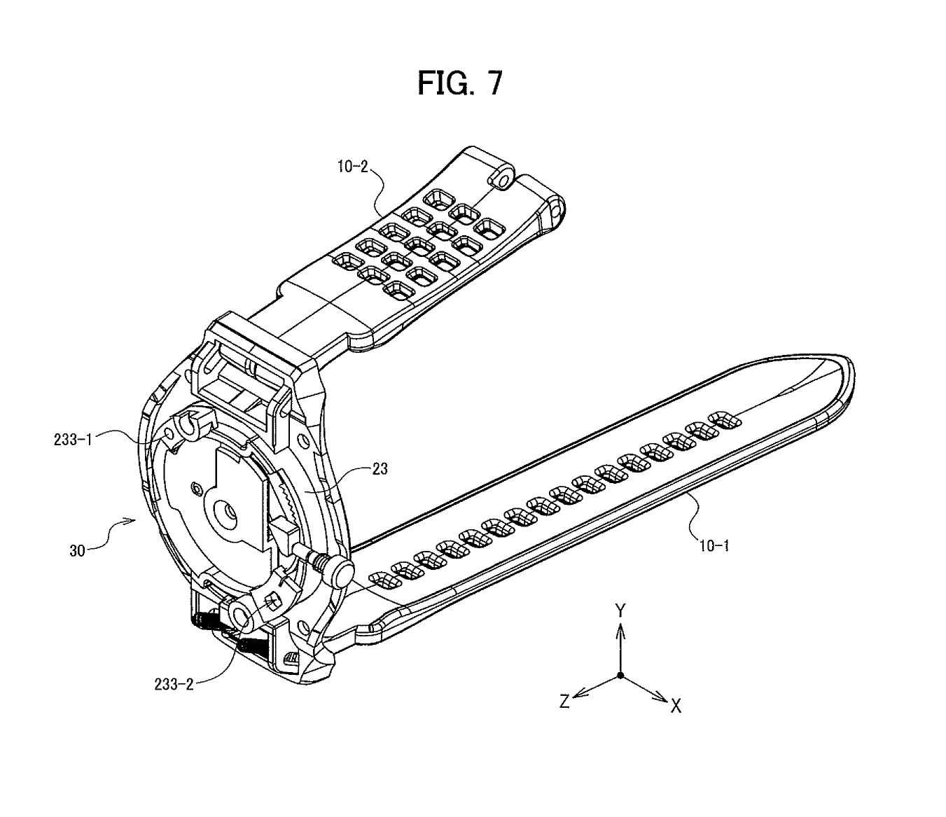

FIG. 6 is a plan view and a side view illustrating the biological information detection device, and FIG. 7 is a perspective view illustrating the biological information detection device. Note that FIGS. 6 and 7 illustrate a state in which the display section 50, the rotary bezel 22, and the like are removed so that the internal structure (particularly the adjustment mechanism 20) of the case 30 can be readily understood. FIG. 6 includes a plan view illustrating the biological information detection device viewed from the side situated in the positive Z-axis direction (i.e., the direction from the subject toward the case in a state in which the user wears the biological information detection device) (see E1), a side view illustrating the biological information detection device viewed from the side situated in the positive Y-axis direction (see E2), a side view illustrating the biological information detection device viewed from the side situated in the positive X-axis direction (see E3), and a side view illustrating the biological information detection device viewed from the side situated in the negative Y-axis direction (see E4).

As illustrated in FIGS. 6 and 7, the biological information detection device has a structure similar to that of a normal wristwatch. The biological information detection device includes the case 30 (main body) and the band 10, and the band 10 includes a first band 10-1 and a second band 10-2. Note that the buckle 14 that includes the locking member 16 is provided to the end of the second band 10-2 that is situated opposite to the case 30 (not illustrated in FIGS. 6 and 7).

Reference numerals 233-1 and 233-2 in FIGS. 6 and 7 indicate the recess that is provided to the ratchet mechanism 23. The protrusion of the rotary bezel 22 is inserted into the recess so that the rotary bezel 22 and the ratchet mechanism 23 engage with each other.

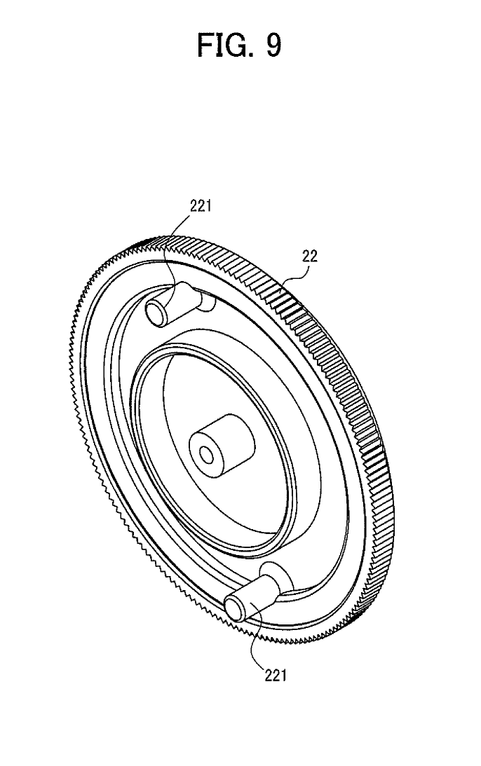

FIG. 8 is a plan view and a side view illustrating the rotary bezel 22, and FIG. 9 is a perspective view illustrating the rotary bezel 22. The plan view (see F1) in FIG. 8 illustrates the rotary bezel 22 viewed from the side situated in the negative Z-axis direction in a state in which the rotary bezel 22 is secured on the case 30 of the biological information detection device. The side view (see F2) in FIG. 8 illustrates the rotary bezel 22 (see F1) viewed from the upper side in FIG. 8. FIG. 9 is a perspective view illustrating the rotary bezel 22 viewed from a viewpoint from which the side of the rotary bezel 22 situated on the side in the negative Z-axis direction can be observed in a state in which the rotary bezel 22 is secured on the case 30.

The rotary bezel 22 includes protrusions 221-1 and 221-2. The protrusions 221-1 and 221-2 engage with the recesses 233-1 and 233-2 of the ratchet mechanism 23 illustrated in FIGS. 6 and 7. The rotary bezel 22 has an approximately disc-like shape (i.e., an approximately cylindrical shape in which the height is smaller than the radius of the circles that form the bottom side and the upper side of the cylinder). For example, convexities and concavities (tooth profile) may be formed on the side of the rotary bezel 22. When convexities and concavities are formed on the side of the rotary bezel 22, the user can easily rotate the rotary bezel 22. The biological information detection device includes the display section 50 (see FIG. 2), and the rotary bezel 22 may be provided at a position that corresponds to the frame of the display section 50. In this case, it may be difficult for the user to observe the information displayed on the display section 50 if the display section 50 is rotated in conjunction with the rotation of the rotary bezel 22. Therefore, it is desirable to configure the rotary bezel 22 so that the rotary bezel 22 can be rotated independently of the display section 50.

Although FIGS. 6 to 9 illustrate an example in which the rotary bezel 22 and the ratchet mechanism 23 are caused to engage with each other using the protrusions 221-1 and 221-2 and the recesses 233-1 and 233-2, the configuration is not limited thereto. When the rotary bezel 22 and the ratchet mechanism 23 are caused to engage with each other using a protrusion and a recess, and the engagement between the rotary bezel 22 and the ratchet mechanism 23 is released by moving the rotary bezel 22 in the positive Z-axis direction (i.e., the direction that intersects the rotation direction and is away from the subject) illustrated in FIG. 5, it is necessary to position the protrusion and the recess when causing the rotary bezel 22 and the ratchet mechanism 23 to engage with each other again (i.e., the user must perform a complex operation). Therefore, teeth-like convexities and concavities may be provided to part of the bottom side (i.e., the side that directly faces the ratchet mechanism 23 and is provided with the protrusions 221-1 and 221-2 (see FIG. 9)) of the rotary bezel 22, and convexities and concavities that can engage with the convexities and concavities of the rotary bezel 22 may be provided to the side of the ratchet mechanism 23 that faces the rotary bezel 22. In this case, it is unnecessary to perform strict positioning when causing the rotary bezel 22 and the ratchet mechanism 23 to engage with each other again after the engagement between the rotary bezel 22 and the ratchet mechanism 23 has been released.

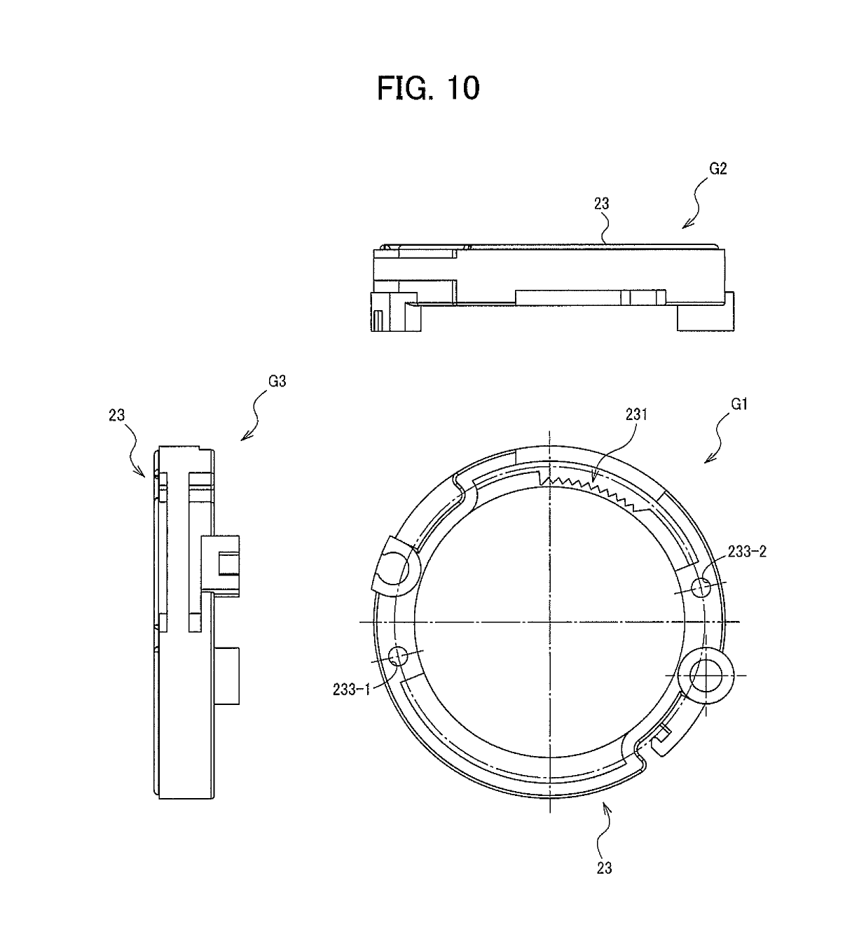

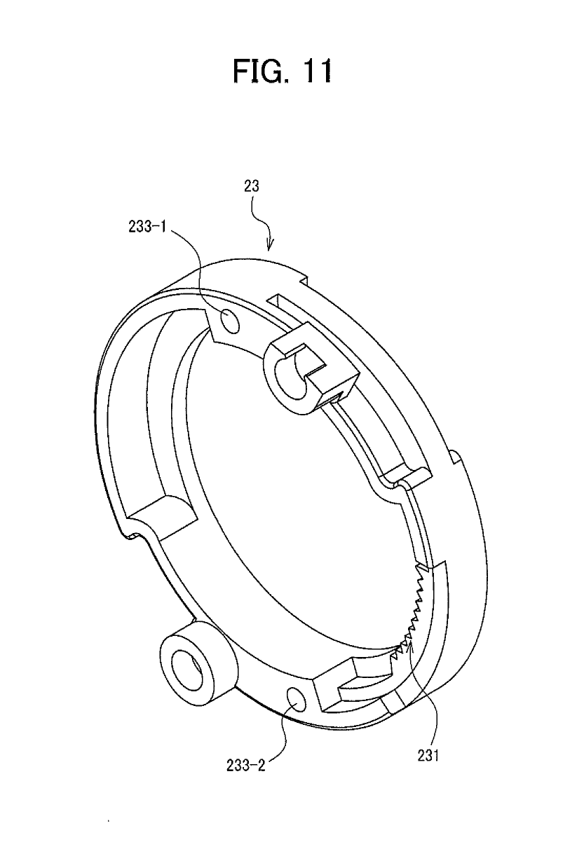

FIG. 10 is a plan view and a side view illustrating the ratchet mechanism 23 excluding the pawl 232, and FIG. 11 is a perspective view illustrating the ratchet mechanism 23 excluding the pawl 232. Reference sign G1 in FIG. 10 indicates a plan view illustrating the ratchet mechanism 23 viewed from the side situated in the positive Z-axis direction. Reference sign G2 indicates a side view illustrating the rotary bezel 22 (see G1) viewed from the upper side in FIG. 10, and reference sign G3 indicates a side view illustrating the rotary bezel 22 (see G1) viewed from the left side in FIG. 10. FIG. 10 is a perspective view illustrating the ratchet mechanism 23 viewed from a viewpoint from which the side of the ratchet mechanism 23 situated on the side in the positive Z-axis direction can be observed. Note that the above direction refers to the direction in a state in which the ratchet mechanism 23 is secured on the case 30.

As illustrated in FIG. 10, the ratchet mechanism 23 includes the recesses 233-1 and 233-2 that are used for the engagement with the rotary bezel 22, and the gear 231 that allows the ratchet mechanism 23 to be rotated only in one rotation direction. Although FIG. 3 (schematic view) illustrates an example in which two ratchet mechanisms 23 are provided, it is also possible to use the ratchet mechanism 23 having the ring-like shape illustrated in FIGS. 10 and 11. The ratchet mechanism 23 is provided to the biological information detection device at the position illustrated in FIG. 7.

FIG. 12 is a plan view illustrating a state in which the ratchet mechanism 23 illustrated in FIGS. 10 and 11 is secured on the case 30. FIG. 12 is a plan view illustrating the biological information detection device viewed from the side situated in the positive Z-axis direction. As illustrated in FIG. 12, the pawl 232 that forms the ratchet mechanism 23 is provided at a position closer to the center of the case 30 (than the mechanism illustrated in FIGS. 10 and 11) in a plane having the same height (i.e., the position in the direction that intersects the display section 50 (i.e., the direction from the subject toward the case 30 (i.e., the position in the Z-axis direction (see FIG. 12)) as that of the mechanism illustrated in FIGS. 10 and 11 and extends along the display section 50.

The pawl 232 can be rotated in the XY plane around a rotation fulcrum 234. The pawl 232 can be rotated to some extent in the direction DR4 (i.e., the clockwise direction in FIG. 12), but the rotation of the pawl 232 in the direction DR5 (counterclockwise direction) is limited since interference with the member (gear 231) illustrated in FIG. 10 occurs.

Therefore, when a force that rotates the gear 231 clockwise in conjunction with the rotary bezel 22 has been applied, the pawl 232 is pushed by the teeth of the gear 231 and moved in the direction DR4, and the ratchet mechanism 23 can be rotated in conjunction with the rotary bezel 22. When a force that rotates the gear 231 counterclockwise has been applied, a force that moves the pawl 232 in the direction DR5 is applied. However, the pawl 232 cannot be moved to a position that does not hinder the rotation of the gear 231, and the rotation of the ratchet mechanism 23 (and the rotary bezel 22) is prohibited.

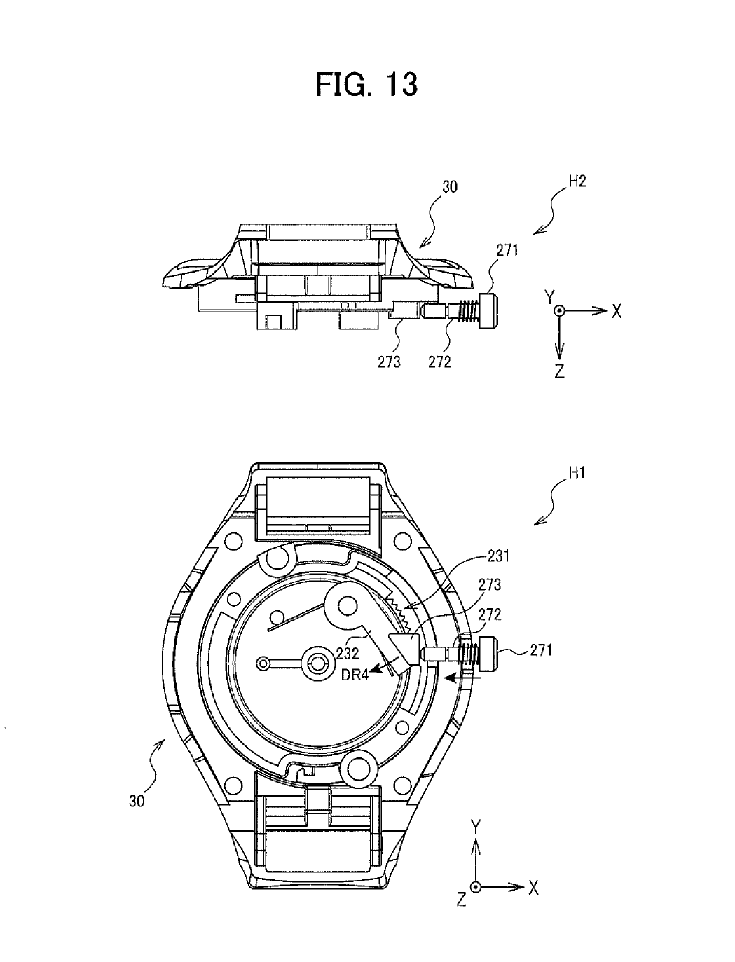

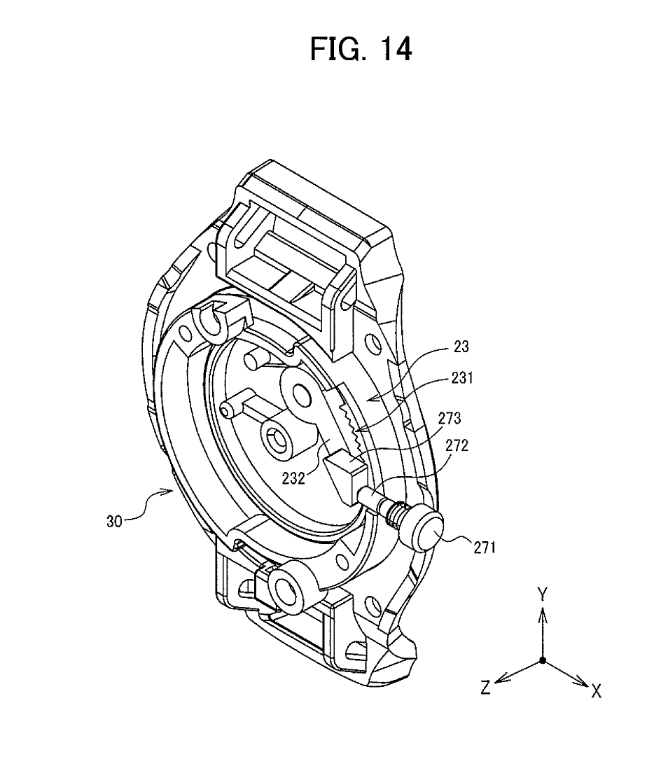

The release state in which the restriction of the rotation direction by the restriction mechanism is released (canceled) has been described above. Note that the release method is not limited to the method that moves the rotary bezel 22 upward. FIGS. 13 and 14 illustrate an example of another release mechanism.

FIG. 13 is a plan view and a side view illustrating the release mechanism, and FIG. 14 is a perspective view illustrating the release mechanism. Reference sign H1 in FIG. 13 indicates a plan view illustrating the biological information detection device viewed from the side situated in the positive Z-axis direction, and reference sign H2 in FIG. 13 indicates a side view illustrating the biological information detection device viewed from the side situated in the positive Y-axis direction. FIG. 14 is a perspective view illustrating the biological information detection device viewed from a viewpoint from which the side of the case 30 situated on the side in the positive Z-axis direction can be observed. The release mechanism may include a release button 271 illustrated in FIGS. 13 and 14, for example. In the example illustrated in FIGS. 13 and 14, the release mechanism includes a release member 273 that comes in contact with the pawl 232, and is moved toward the center of the case 30 in conjunction with the release button 271 when the release button 271 is pressed.

In the example illustrated in FIGS. 13 and 14, when the release button 271 is pressed, a rod-like member 272 that is connected to the release button 271 is forced toward the center of the case 30, and the release member 273 is forced toward the center of the case 30 in conjunction with the rod-like member 272. When the release member 273 is moved toward the center of the case 30, the pawl 232 that comes in contact with the release member 273 is rotated in the direction DR4. Specifically, the gear 231 and the pawl 232 of the ratchet mechanism 23 disengage from each other when the release button 271 has been pressed, and the ratchet mechanism 23 no longer limits the rotation direction (i.e., the release state is implemented). It suffices that the release mechanism be able to implement a state in which the restriction (on the rotation direction) by the restriction mechanism is released (canceled). The specific structure of the release mechanism may be modified in various ways.

### 4. Modifications

As described above, the adjustment mechanism 20 according to one embodiment of the invention may be provided to the case 30. In this case, it is natural to use the rotary bezel 22 as the rotary member 21. Since the rotary bezel 22 is used for a common wristwatch and the like, the user can use the rotary bezel 22 without confusion.

Note that the adjustment mechanism 20 according to one embodiment of the invention may be provided at a position differing from the case 30. As illustrated in FIG. 15, the band 10 may include the first band 10-1 and the second band 10-2, the case 30 may be provided between a first end BE11 of the first band 10-1 and a first end BE21 of the second band 10-2, and the adjustment mechanism 20 may be provided between a second end BE12 of the first band 10-1 that differs from the first end BE11, and a second end BE 22 of the second band 10-2 that differs from the first end BE21, for example.

For example, the biological information detection device may include a connection section 60 (buckle) that is provided between the second end BE12 of the first band 10-1 and the second end BE22 of the second band 10-2, and the adjustment mechanism 20 may be provided to the connection section 60. The connection section 60 may be unremovably connected to one of the first band 10-1 and the second band 10-2, and configured so that the other of the first band 10-1 and the second band 10-2 can be secured using a locking member or the like (so as to be removal and adjusted in position). Alternatively, the connection section 60 may be connected to both the first band 10-1 and the second band 10-2 so that the first band 10-1 and the second band 10-2 are not removed from the connection section 60.

In either case, the connection section 60 has a space for holding (pulling) the band 10, and includes the ratchet mechanism 23 and the like. In this case, the rotary member 21 need not be formed in the shape of a bezel. Various rotatable operation sections can be used as the rotary member 21. For example, the rotary operation section 28 illustrated in FIG. 15, or an operation section having a structure similar to that of the rotary operation section 28 may be used as the rotary member 21. At least one of the first band 10-1 and the second band 10-2 is pulled in the first direction (in which the biological information detection section 40 (sensor unit) comes in contact with the subject) by rotating the rotary operation section 28 in a given direction (e.g., clockwise). In the example illustrated in FIG. 15, the first direction refers to the direction toward the member that is provided with the adjustment mechanism 20 such as the connection section 60 (i.e., the direction opposite to the direction toward the case 30).

According to this configuration, it is possible to provide the adjustment mechanism 20 at a position differing from the case 30. Since the biological information detection section 40 (sensor unit) and electronic parts (e.g., a main board and an IC provided to the main board) that implement the processing section 200 and the like are provided to the case 30, the size of the case 30 (and the entire biological information detection device) may increase when the adjustment mechanism 20 is also provided inside the case 30 (see FIG. 2 and the like). According to the modification illustrated in FIG. 15, since the adjustment mechanism 20 can be provided separately from the case 30, it is possible to reduce the size of the case 30.

As described above, the connection section 60 may be connected to both the first band 10-1 and the second band 10-2 so that the first band 10-1 and the second band 10-2 are not removed from the connection section 60. In this case, a mechanism that is used to roughly adjust the inner diameter of the band 10 may not be provided, and the band 10 may be adjusted by (using) the adjustment mechanism 20.

For example, the biological information detection device is designed so that the band 10 is loosened (i.e., increased in inner diameter) to such an extent that the band 10 can be fitted to the wrist of the user when the band 10 is not pulled by the adjustment mechanism 20, and the band 10 is fastened using only the adjustment mechanism 20. Alternatively, the band 10 may be formed using an elastic member so that the band 10 can be easily fitted to the wrist of the user. In either case, it is possible to implement an appropriate wearing state using only the adjustment mechanism 20 without performing an adjustment using the fitting holes 12, the locking member 16, and the like as long as the pulling width of the band 10 by the adjustment mechanism 20 is sufficiently large.

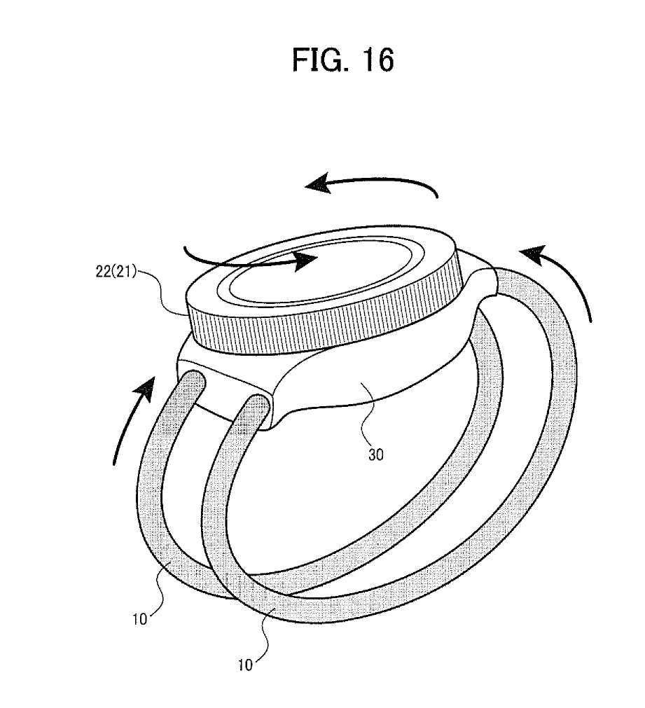

The adjustment mechanism 20 may be provided to the case 30, and the band 10 may be adjusted by the adjustment mechanism 20 without using the fitting holes 12 and the like. FIG. 16 illustrates a specific example. According to the configuration illustrated in FIG. 16, it is unnecessary to divide the band 10 into two sections that are connected to one end and the other end of the case 30, and the ends of the case 30 are connected by one band 10. The adjustment mechanism 20 pulls the band 10 in the first direction (i.e., the direction toward the case 30) when the rotary member 21 (e.g., rotary bezel 22) that is provided to the case 30 is rotated so that the degree of fastening of the band 10 is adjusted. In this case, since the band 10 is loosened to a maximum extent (i.e., the band 10 cannot be further loosened) in a state in which the adjustment mechanism 20 is not operated, it is desirable to set the inner diameter (length) of the band 10 so that the band 10 can be fitted to the wrist in a state in which the band 10 is loosened to a maximum extent. The band 10 that is loosened to a maximum extent is fastened by the adjustment mechanism 20.

In the example illustrated in FIG. 16, since the adjustment mechanism 20 is provided to the case 30, it is necessary to take account of the size of the case 30 (see above). As illustrated in FIG. 16, the band 10 may be implement using a wire-like member. For example, the adjustment mechanism 20 may pull the wire-like band 10 directly instead of separately providing the wire 26 and the band 10 (see FIG. 3). According to this configuration, since the thin wire-like band 10 is pulled by the adjustment mechanism 20, a large space is not required even when the band 10 is pulled into the case 30.

When the thin wire-like band 10 is used, the user may feel pain when the band 10 is fastened, for example. Therefore, it is desirable to implement the band 10 using a plurality of wire-like members (see FIG. 16), or provide a cushion member.

Although only some embodiments of the invention have been described in detail above, those skilled in the art will readily appreciate that many modifications are possible in the embodiments without materially departing from the novel teachings and advantages of the invention. Accordingly, all such modifications are intended to be included within scope of this invention. Any term cited with a different term having a broader meaning or the same meaning at least once in the specification and the drawings can be replaced by the different term in any place in the specification and the drawings. The configuration and the operation of the biological information detection device are not limited to those described above in connection with the embodiments. Various modifications and variations may be made of those described above in connection with the embodiments.

What is claimed is:

1. A biological information detection device comprising:
a case that is provided with a sensor that detects biological information about a subject;
a band that secures the case on the subject; and
an adjustment mechanism that moves the band in a first direction when a rotary member that is supported by the case has been rotated, the first direction being a direction in which a side of the case that is situated opposite to the subject comes in contact with the subject.

2. The biological information detection device as defined in claim 1,
the adjustment mechanism including a restriction mechanism that restricts movement of the band in a second direction that is a direction opposite to the first direction.

3. The biological information detection device as defined in claim 2,
the adjustment mechanism including a wire that is connected to the band, and is at least either moved or deformed due to the rotation of the rotary member.

4. The biological information detection device as defined in claim 2,
the rotary member being a rotary bezel, and
the restriction mechanism being a ratchet mechanism that engages with the rotary bezel, and restricts the movement of the band in the second direction.

5. The biological information detection device as defined in claim 4,
the adjustment mechanism including a wire that connects the ratchet mechanism and the band, and being configured so that the ratchet mechanism is rotated in a first rotation direction in conjunction with the rotary bezel when the rotary bezel is rotated in the first rotation direction so that the wire moves the band in the first direction.

6. The biological information detection device as defined in claim 4,
the ratchet mechanism disengaging from the rotary bezel so that the band can be moved in the second direction when the rotary bezel has been moved upward in a direction that intersects a rotation plane of the rotary bezel.

7. The biological information detection device as defined in claim 4,
the rotary bezel being movable in a direction that intersects a rotation plane of the rotary bezel so that the band can be moved in the second direction.

8. The biological information detection device as defined in claim 4,
the band including a plurality of fitting holes, and a locking member that is fitted into a fitting hole among the plurality of fitting holes to lock the band, and
a moving amount of the band corresponding to one tooth of a gear that is included in the ratchet mechanism being smaller than an interval between the plurality of fitting holes.

9. The biological information detection device as defined in claim 1,
the adjustment mechanism being provided to the case.

10. The biological information detection device as defined in claim 1,
the band including a plurality of fitting holes, and a locking member that is fitted into a fitting hole among the plurality of fitting holes to lock the band, and
the adjustment mechanism implementing fastening of the band between a state in which the locking member is fitted into a first fitting hole among the plurality of fitting holes, and a state in which the locking member is fitted into a second fitting hole among the plurality of fitting holes that is situated adjacent to the first fitting hole.

11. The biological information detection device as defined in claim 10,
the adjustment mechanism being able to move the band in the first direction at an interval smaller than an interval between the plurality of fitting holes.
